(19) United States
(12) Patent Application Publication
(10) Pub. No.: US 2010/0042176 A1
(43) Pub. Date: Feb. 18, 2010

(54) TEMPORAL-BASED CARDIAC CAPTURE THRESHOLD DETECTION

(75) Inventor: Jeffery D. Snell, Chatsworth, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/190,538

(22) Filed: Aug. 12, 2008

(51) Int. Cl. A61N 1/362

(52) U.S. Cl. 607/28

(57) ABSTRACT

A cardiac capture threshold may be determined using a test pulse and a backup pulse. Here, delivery of a test pulse is followed almost immediately by a non-conditional backup pulse of sufficient energy such that the backup pulse should always capture in the event the test pulse does not capture. The timing of the evoked response that follows the backup pulse may then be used to determine whether the test pulse or the backup pulse captured the cardiac tissue. In some embodiments morphology discrimination may be employed to determine whether an evoked response was triggered by the test pulse or the backup pulse. In some embodiments timing information associated with one or more features of the evoked response may be analyzed to determine whether an evoked response was triggered by the test pulse or the backup pulse.

### TECHNICAL FIELD

This application relates generally to implantable cardiac stimulation devices, and to determining a cardiac capture threshold.

### BACKGROUND

When a person's heart does not function normally due to, for example, a genetic or acquired condition, various treatments may be prescribed to correct or compensate for the condition. For example, pharmaceutical therapy may be prescribed for a patient or an implantable cardiac device may be implanted in the patient to improve the function of the patient's heart.

In a healthy heart, contractions occur first in the muscles associated with the atrial chambers of the heart, followed by contractions in the muscles associated with the larger ventricular chambers of the heart. In this way, the atria assist in the filling of the ventricular chambers with blood returning from the veins. This increases the end-diastolic volume increasing the stroke volume to enable the ventricles to more efficiently pump blood to the arteries.

Given the interaction of these chambers, efficient operation of the heart is predicated on each of the chambers operating in a proper timing sequence and having contractions that pump a sufficient amount of blood from the chamber. For example, during contraction the right atrium chamber should pump enough blood to optimally “fill” the right ventricle chamber. Moreover, this should occur immediately before the right ventricle begins to contract. In this way, the heart may efficiently pump blood on a repetitive basis.

A healthy heart repetitively contracts in the above described manner in response to the generation and conduction of native electrical signals in the heart. These electrical signals are generated in and conducted through the heart during every beat of the heart. Activity for a given beat begins with the generation of a signal in a sinus node of the heart. This signal causes contraction to begin first in the atria. The signal from the sinus node propagates via a conduction system to an atrioventricular (“A-V”) node. The signal is delayed for a short period of time (e.g., 120-200 milliseconds) within the AV node allowing the atria to contract to help to fill the ventricles. The signal then propagates from the A-V node through the bundle of His to the left and right ventricles via a specialized conduction system. Contraction in each ventricle commences in a coordinated manner when the signal “reaches” the respective muscle fibers in the ventricle.

In a diseased or otherwise unhealthy heart, there may be a disruption or abnormality in the generation and/or propagation of these signals. For example, in some patients the atria may generate signals in a sporadic manner or there may be a blockage that prevents the signal from the sinus node from reaching the ventricles in a normal manner. In either of these cases, the atrial-ventricular timing may be compromised resulting in inefficient operation or failure of the heart. In other patients, the activation of the main pumping chamber, the left ventricle, is abnormal compromising the coordination of left ventricular contraction and thus compromising cardiac efficiency.

Under certain circumstances, an implantable cardiac device (e.g., a pacemaker) may compensate for abnormal operation of a heart by stimulating (e.g., pacing) one or more of the atria and/or ventricles. To stimulate the heart, a typical implantable cardiac device generates a pulse which is applied to the heart via one or more electrodes implanted in the heart (e.g., in ventricular or atrial chambers). This pulse is generated with sufficient energy to cause the heart to contract in much the same way as the native electrical signals discussed above cause the heart to contract.

To provide appropriate timing for the generation of electrical signals, an implantable cardiac device may sense signals in the heart. For example, when the sinus node is not generating signals in a regular manner, the implantable cardiac device may sense electrical signals in the atria to detect whether the atria are being activated each time a beat is expected to commence. If a signal is not detected, the implantable cardiac device may immediately apply a pacing pulse to, for example, the right atrium. In this way, the implantable cardiac device may stimulate the heart at the appropriate times, as necessary, in an attempt to maintain efficient operation of the heart.

In conjunction with pacing operations, an implantable cardiac device may perform a capture threshold test to ensure that pacing pulses are generated with an amount of energy that is sufficient to induce activation of cardiac muscle tissue (conventionally referred to as “capture”) yet does not unduly waste power. In this way, the regular rhythm of the heart may be maintained without unnecessarily diminishing the useful life of the battery that powers the implantable cardiac device. In practice, such tests are typically performed on a regular basis because the amount of energy required to cause capture may change over time.

A typical capture threshold test involves applying a test pacing pulse, determining whether the test pulse caused capture and, as necessary, adjusting the amplitude of the test pulse then repeating the procedure until a minimum amplitude level (with a sufficient safety margin) is found that achieves capture.

To maintain the regular rhythm of the heart when performing these tests, provisions may be made to ensure that a beat is not skipped when the amplitude level of the test pulse is not sufficient to cause capture. For example, in the event the test pulse does not cause capture, a backup pacing pulse having a magnitude that is expected to cause capture may be applied to the heart. In practice, however, it may not always be possible to accurately determine whether the test pulse resulted in capture before a decision needs to be made as to whether a backup pulse is needed. For example, to avoid inducing fibrillation in the heart, a backup pulse is typically applied no more than a short time (e.g., 40 milliseconds) after application of a test pulse. However, the signal level of the initial portion (e.g., the first 40 milliseconds) of the evoked response to the pacing pulse may be relatively small in the atria during this short period of time. As a result, this initial portion of the evoked response may not be readily distinguishable from other signals (e.g., polarization-induced signals) in the heart. Consequently, an implantable cardiac device that uses the above technique may generate backup pulses when they are not needed or may not generate backup pulses when they are needed.

Another capture detection technique attempts to detect a “side effect” of atrial capture. For example, if the atrium is captured, the resulting depolarization may be conducted to the ventricle such that a contraction in the ventricle is a “side effect” of capturing the atrium. In other words, if the atrium was not captured then the ventricular contraction would not have occurred at the time expected, based on the time the atrial test pulse was delivered. In practice, however, this technique may not be desirable because the detection of the “side effect” may occur too late to enable application of a backup pulse to ensure cardiac activity. Moreover, such a technique may not work for patients having some form of bundle branch block.

Another capture detection technique involves determining whether the sinus node exhibits a compulsory pause in response to overdrive pacing. Here, P-waves may be monitored to determine whether the rhythm of the P-waves changes in response to the pacing pulse, thereby indicating that a pacing pulse has captured. In practice, effective use of this technique may require that the patient have a high enough intrinsic cardiac rate.

Another capture technique utilizes separate vectors for pacing and sensing. For example, separate electrode areas may be provided at the case of an implanted cardiac device and each of these electrodes may be used with different electrodes (e.g., tip or ring) on the distal end of an implanted lead. In this way, some of the signal detection problems caused by depolarization may be reduced. In practice, however, an implantable cardiac device using this approach may still have difficulty detecting relatively small evoked responses. Moreover, the complexity added by this approach may not be desirable in some applications.

In view of the above, there is a need for improved techniques for determining capture threshold.

### SUMMARY

A summary of sample aspects of the disclosure or sample embodiments of an apparatus constructed or a method practiced according to the teaching herein follows. For convenience, one or more of such aspects or embodiments may be referred to herein simply as “some aspects” or “some embodiments.”

The disclosure relates in some aspects to determining a cardiac capture threshold using a test pulse and a backup pulse. Here, delivery of a test pulse is followed almost immediately by a non-conditional backup pulse of sufficient energy to cause capture. Thus, if the test pulse is effective in capturing the cardiac tissue then the backup pulse will be of no effect. Conversely, if the test pulse does not capture the cardiac tissue then the backup pulse will likely cause capture.

In accordance with some aspects of the disclosure the above relationship may be exploited such that a responsive cardiac signal (e.g., an evoked response) that follows the backup pulse may be analyzed to determine whether the test pulse or the backup pulse captured the cardiac tissue. For example, the timing between the test pulse and the backup pulse may be set such that the timing of a resulting evoked response indicates whether the test pulse or the backup pulse triggered the evoked response. In particular, an evoked response that occurs at a given point in time may indicate capture by a first pulse (e.g., the test pulse) while an evoked response that occurs at a later point in time may indicate capture by a later occurring pulse (e.g., the backup pulse). This timing information may be monitored while changing the magnitude (e.g., amplitude) of the test pulse to effectively determine the capture threshold. For example, a change relating to which pulse triggered the evoked response (e.g., caused by a change in the amplitude of the test pulse that crossed a physical capture threshold) may be identified by detecting a time shift between the corresponding evoked responses.

Advantageously, because the evoked response is not used to determine whether to generate the backup pulse, a portion (e.g., a beginning portion) or the entirety of the cardiac response signal that follows the backup pulse may be analyzed to determine whether the test pulse or the backup pulse captured cardiac tissue. For example, in a capture threshold test for atrial pacing, a portion or all of a P-wave (typically 200 milliseconds in length) may be utilized to determine which pulse resulted in capture. Similarly, in a capture threshold test for ventricular pacing, a portion or all of an R-wave may be utilized to determine which pulse resulted in capture. In some embodiments this signal may be acquired via a global signal sensing technique utilizing, for example, a tip-to-can vector. In this way, implantable cardiac device may sense over a large area thereby acquiring a relatively large response signal. Through the use of such techniques, capture may be detected even when the initial portion of the evoked response for a given patient has a relatively small signal level.

In some embodiments morphology discrimination is employed to determine whether an evoked response was triggered by the test pulse or the backup pulse. For example, one or more morphology templates may be generated based on a typical (e.g. average) evoked response that follows the backup pulse. Here, different sets of morphology information will correspond to when the test pulse resulted in capture versus when the backup pulse resulted in capture. For example, the information associated with the backup pulse may correspond to a waveform that is shifted in time with respect to a corresponding waveform associated with the test pulse. Accordingly, morphology information based on (e.g., representative of) a current evoked response may be compared to the morphology template(s) to determine which pulse triggered the evoked response.

In some embodiments timing information associated with one or more features of the evoked response may be analyzed to determine whether an evoked response was triggered by the test pulse or the backup pulse. For example, baseline timing information relating to characteristics such as the time of a maximum amplitude (e.g., a peak in the signal), a maximum slope, a minimum slope, or some other suitable characteristic may be generated based on a typical (e.g., an average) evoked response that follows the backup pulse. Here, different time values will correspond to when the test pulse resulted in capture and when the backup pulse resulted in capture. For example, the time of a given feature resulting from backup pulse capture will be later, relative to the backup pulse, than the time of the corresponding feature resulting from test pulse capture. Accordingly, corresponding timing information based on (e.g., derived from) a current evoked response may be compared to the baseline timing information to determine which pulse triggered the evoked response.

The above techniques may be utilized in various ways. For example, one or more of the above techniques may be used to periodically measure the capture threshold, or to periodically measure the capture threshold and then adjust the normal pacing energy. One or more of the above techniques may be used to periodically measure the capture threshold in conjunction with a beat-by-beat capture monitoring algorithm. The above techniques may be used to pace in the right atrium, the right ventricle, the left ventricle, any other suitable location, or any combination of these locations. The above techniques may be used with any pacing scheme that operates by pacing an electrode that is in direct contact with the cardiac muscle or that is not in direct contact with the cardiac muscle.

### BRIEF DESCRIPTION OF THE DRAWINGS

These and other features, aspects and advantages that may relate to the invention will be more fully understood when considered with respect to the following detailed description, appended claims and accompanying drawings, wherein:

FIG. 1 is a flow chart of an embodiment of operations that may be performed to determine a capture threshold;

FIG. 2 is a simplified block diagram of an embodiment of an apparatus for determining a capture threshold;

FIG. 3, including FIGS. 3A and 3B, depicts simplified pacing and evoked response waveforms;

FIG. 4 is a flow chart of an embodiment of operations that may be performed to determine a capture threshold;

FIG. 5 is a flow chart of an embodiment of operations that may be performed to identify a pulse that resulted in capture;

FIG. 6 is a flow chart of an embodiment of operations that may be performed to identify a pulse that resulted in capture;

FIG. 7 is a simplified diagram of an embodiment of an implantable stimulation device in electrical communication with one or more leads implanted in a patient's heart for sensing conditions in the patient, delivering therapy to the patient, or providing some combination thereof; and

FIG. 8 is a simplified functional block diagram of an embodiment of an implantable cardiac device, illustrating basic elements that may be configured to sense conditions in the patient, deliver therapy to the patient, or provide some combination thereof.

In accordance with common practice the various features illustrated in the drawings may not be drawn to scale. Accordingly, the dimensions of the various features may be arbitrarily expanded or reduced for clarity. In addition, some of the drawings may be simplified for clarity. Thus, the drawings may not depict all of the components of a given apparatus (e.g., device) or method. Finally, like reference numerals may be used to denote like features throughout the specification and figures.

### DETAILED DESCRIPTION

The invention is described below, with reference to detailed illustrative embodiments. It will be apparent that the invention may be embodied in a wide variety of forms, some of which may appear to be quite different from those of the disclosed embodiments. Consequently, the specific structural and functional details disclosed herein are merely representative and do not limit the scope of the invention. For example, based on the teachings herein one skilled in the art should appreciate that the various structural and functional details disclosed herein may be incorporated in an embodiment independently of any other structural or functional details. Thus, an apparatus may be implemented or a method practiced using any number of the structural or functional details set forth in any disclosed embodiment(s). Also, an apparatus may be implemented or a method practiced using other structural or functional details in addition to or other than the structural or functional details set forth in any disclosed embodiment(s).

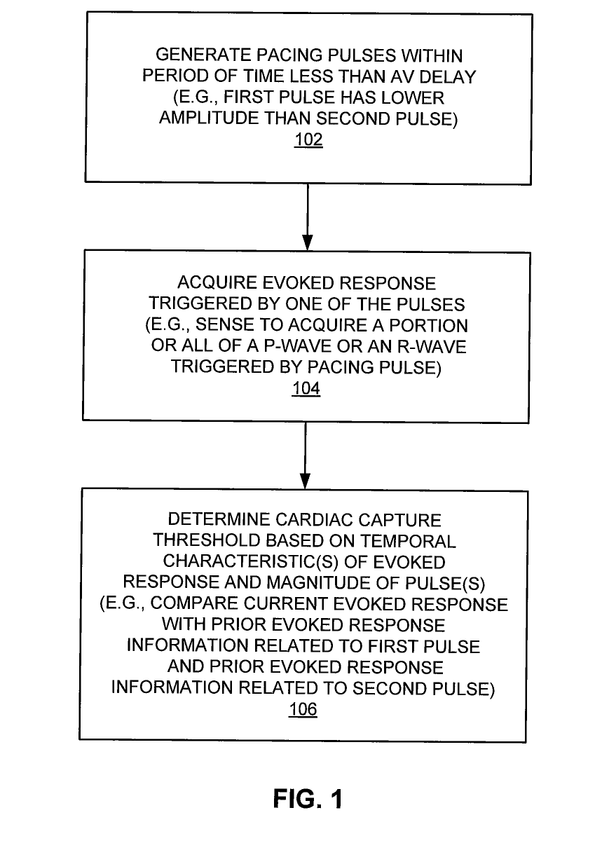

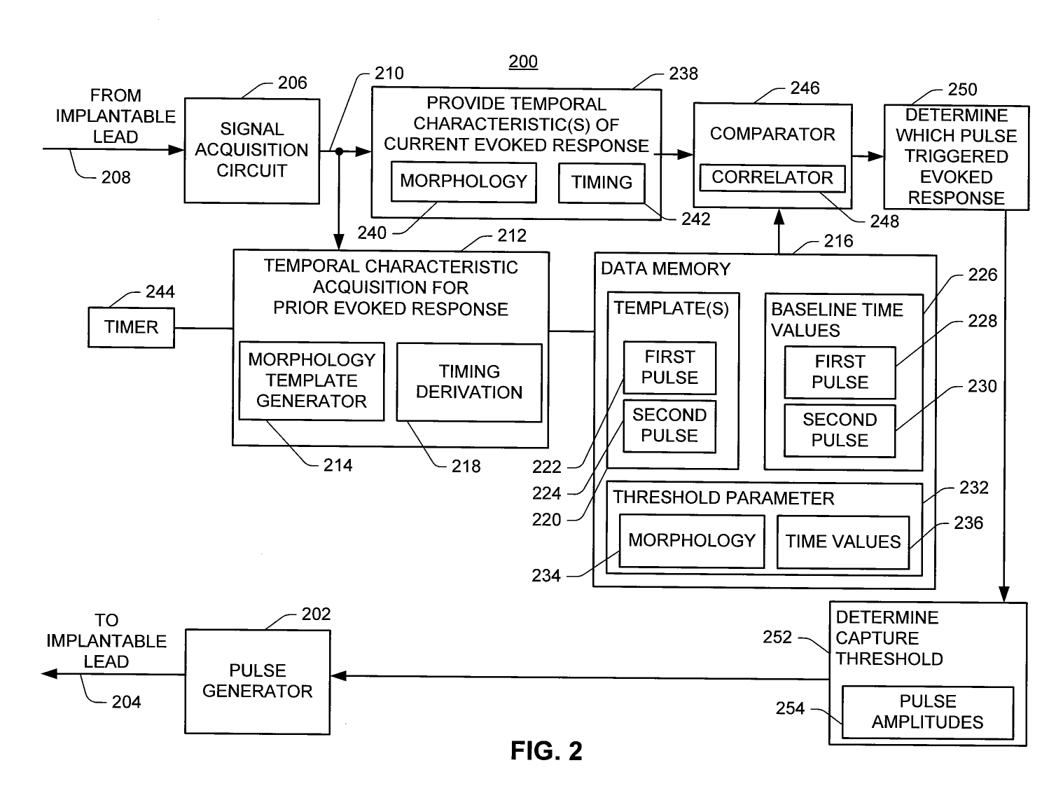

FIG. 1 illustrates an embodiment of several sample operations that may be performed to determine a cardiac capture threshold. The operations of FIG. 1 and other related operations may be performed, for example, by an implantable cardiac device including functional components similar to those illustrated in the system 200 of FIG. 2.

As will be discussed in more detail below, during normal pacing operations (e.g., bradycardia pacing) the implantable cardiac device will collect and analyze information representative of evoked response signals that follow pacing pulses. Typically, these operations will not be performed when test pacing pulses are being applied or during execution of other algorithms that might interfere with the pacing. In the event there are no times of normal pacing, the system may periodically overdrive the heart to acquire evoked response signals.

In a typical implementation the implantable cardiac device will collect several samples of the evoked response signals on a regular basis and process these signals to generate a morphology template or other baseline information representative of a typical (e.g., average) evoked response. This information may then be used during the capture threshold test of FIG. 1.

As represented by block 102, a pulse generator 202 (FIG. 2) may be adapted to generate several (e.g., two or more) pacing pulses within a relatively short period of time. For example, the pulse generator 202 may generate a test pulse followed by a non-conditional backup pulse. Here, a determination as to whether the test pulse achieved capture is not made before the backup pulse is generated. Rather, both pulses are generated for each of the iterations of the capture threshold test. As represented by line 204 in FIG. 2, the generated pulses may be coupled to an implantable lead (e.g., an intravenous lead, an epicardial lead, etc., not shown in FIG. 2) that delivers the pulses to one or more electrodes positioned at a designated pacing site within or on a patient's heart.

In accordance with conventional practice, the amplitude of the test pulse is varied over different iterations of the capture threshold test to determine (or approximately determine) a minimum pulse amplitude that results in capture. In addition, the amplitude of the backup pulse is selected such that in the event a test pulse does not capture, application of the backup pulse is expected to capture. For example, the amplitude of the backup pulse may be fixed at an amplitude (e.g., 4.5 volts) that was previously known to cause capture. Alternatively, the amplitude of the backup pulse may be maintained a level that is more than the amplitude level of the test pulse by a fixed amount (e.g., 1 volt). In this case, the amplitude of the backup pulse may be set at the commencement of the capture threshold test to an amplitude that is expected to capture.

The test and backup pulses may be spaced sufficiently close in time such that an evoked response following the backup pulse may be effectively detected. For example, in some embodiments the backup pulse may follow the test pulse by no more that a typical atrioventricular conduction delay (A-V delay) time of a patient (e.g., on the order of 120 milliseconds or less). In this way, any signal sensing that commences after the backup pulse is generated may not be affected by electrical signal activity in another chamber. For example, with atrial pacing it may be desirable sense in the atria over a period of time that occurs before an R-wave is generated in the ventricles in response to a P-wave triggered by the test pulse. In a typical implementation the backup pulse will follow the test pulse by a period of time that is less than 60 milliseconds (e.g., on the order of 40-50 milliseconds). In some aspects the backup pulse may follow the test pulse by a period of time that is short enough to ensure that atrial fibrillation is not induced by pacing into an atrial repolarization. For example, a maximum time period may be defined to be 180-300 milliseconds after the leading edge of the P-wave.

As represented by block 104 in FIG. 1, the implantable cardiac device senses for cardiac signals after the generation of one of the pulses (e.g., the backup pulse) to acquire, when applicable, an evoked response that was triggered by one of the pulses. For example, in FIG. 2 a signal acquisition circuit 206 may be coupled (as represented by line 208) to the implantable lead to receive signals from one or more electrodes positioned at a designated sensing site. In a typical implementation the signal acquisition circuit 206 generates intracardiac electrogram (“IEGM”) information using, for example, conventional techniques. This IEGM information may then be stored in a data memory for later use.

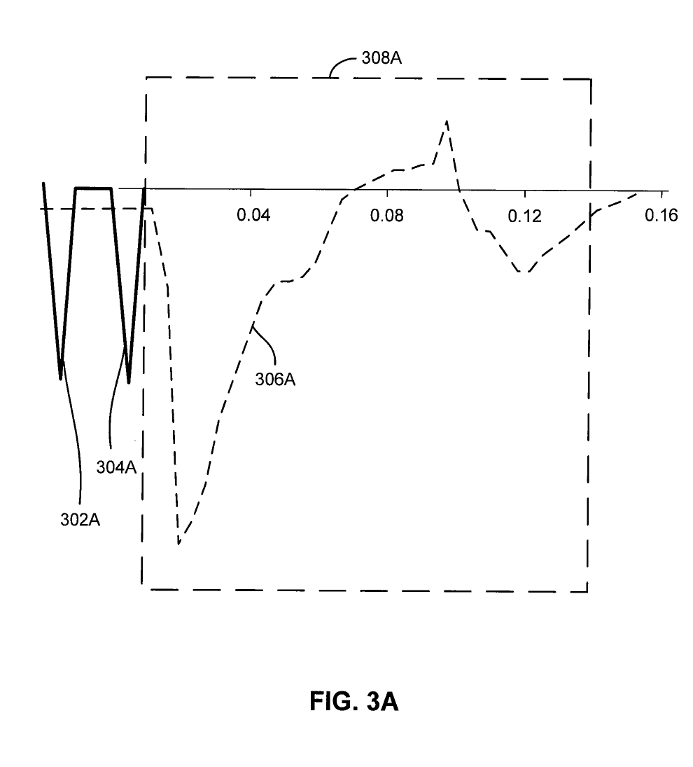

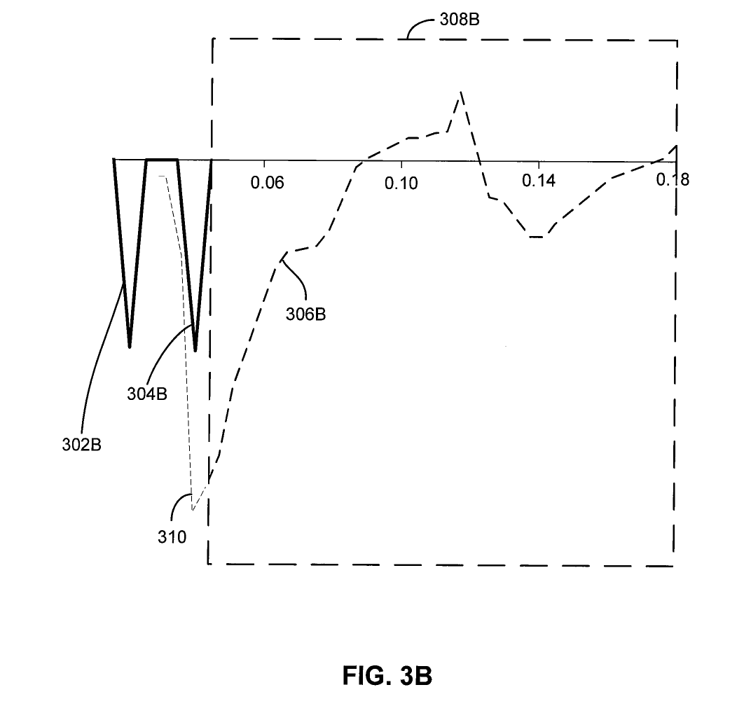

FIG. 3A illustrates a simplified example of a test pulse 302A, a backup pulse 304A, and an evoked response 306A (e.g., a P-wave). In this example the backup pulse 304A captured because the test pulse 302A did not capture. Hence, the evoked response 306A commences after the generation of the backup pulse 304A.

In contrast, FIG. 3B illustrates an example where a test pulse 302B resulted in capture. Thus, in FIG. 3B an evoked response 306B commences after the generation of the test pulse 302B. Here, it may be observed that the evoked response 306B is shifted in time (i.e., is earlier in time) with respect to the evoked response 306A.

FIGS. 3A and 3B also illustrate two examples of evoked response detection windows 308A and 308B. In these examples the detection window commences after the generation of the backup pulse. In the event the test pulse captured, a leading portion of the evoked response will be effectively hidden by a blanking period associated with the backup pulse. For example as shown in FIG. 3B a leading portion 310 of the evoked response 306B is outside of (i.e., precedes) the detection window 308B.

In these examples the detection window is defined to be long enough to acquire all or substantially all of the evoked response. Consequently, a relatively large amount of information may be provided to the implantable cardiac device for use in determining which pulse triggered the evoked response. It should be appreciated that in some implementations only a portion (e.g., a beginning portion) of the evoked response may be acquired at block 104.

As represented by block 106 in FIG. 1, the implantable cardiac device uses the current evoked response information to determine a capture threshold. In particular, one or more temporal characteristics associated with the evoked response may be analyzed to determine whether the test pulse or the backup pulse trigger the evoked response.

The current evoked response may be compared with the prior evoked response information as discussed above to determine whether the timing of the current evoked response corresponds to a typical evoked response triggered by the test pulse (e.g., evoked response 306B) or corresponds to a typical evoked response triggered by the backup pulse (e.g., evoked response 306A). Here, temporal characteristics of the current evoked response may be compared to temporal characteristics of the prior evoked response through the use of morphology templates or baseline timing information.

In some embodiments morphology information representative of the current evoked response may be compared with one or more morphology templates representative of a typical evoked response. As discussed above in conjunction with FIG. 3, the current evoked response observed in the detection window will be shifted in time depending on which pulse triggered a corresponding contraction. Consequently, the morphology information for the current evoked response may be compared with a first set of morphology information that corresponds to capture by the test pulse. In addition, the morphology information for the current evoked response may be compared with a second set of morphology information that corresponds to capture by the backup pulse. With reference to the example of FIG. 3, the first set of morphology information may correspond to a period of time that is similar to the period of time covered by detection window 308B. Similarly, the second set of morphology information may correspond to a period of time that is similar to the period of time covered by detection window 308A.

In some embodiments the evoked response may be compared with baseline timing information relating to one of more features of the evoked response. For example, the baseline timing information may comprise a time at which a typical (e.g., average) evoked response reaches maximum amplitude. Similarly, the baseline information may comprise a time at which a maximum slope and/or a minimum slope occurs in a typical evoked response. It should be appreciated that these are but a few examples of temporal characteristics of an evoked response that may be employed in accordance with the teachings herein.

In a similar manner as discussed above, corresponding timing information derived from the current evoked response will be shifted in time in the detection window depending on which pulse resulted in capture. Consequently, the timing information (e.g., maximum amplitude, maximum slope, and minimum slope) from the current evoked response may be compared with a first set of baseline timing information that corresponds to capture by the test pulse and also compared with a second set of baseline timing information that corresponds to capture by the backup pulse.

Based on the results of the above comparisons, the implantable cardiac device may determine which pulse triggered the evoked response. Thus, in the event it is determined that a test pulse did not capture, the implantable cardiac device may increase the amplitude of the test pulse and then repeat the operations of FIG. 1 as necessary to determine a capture threshold. In accordance with conventional techniques, a suitable capture threshold may be determined based on the lowest amplitude value of the test pulse that resulted in capture, adjusted as necessary to provide a sufficient margin of safety.

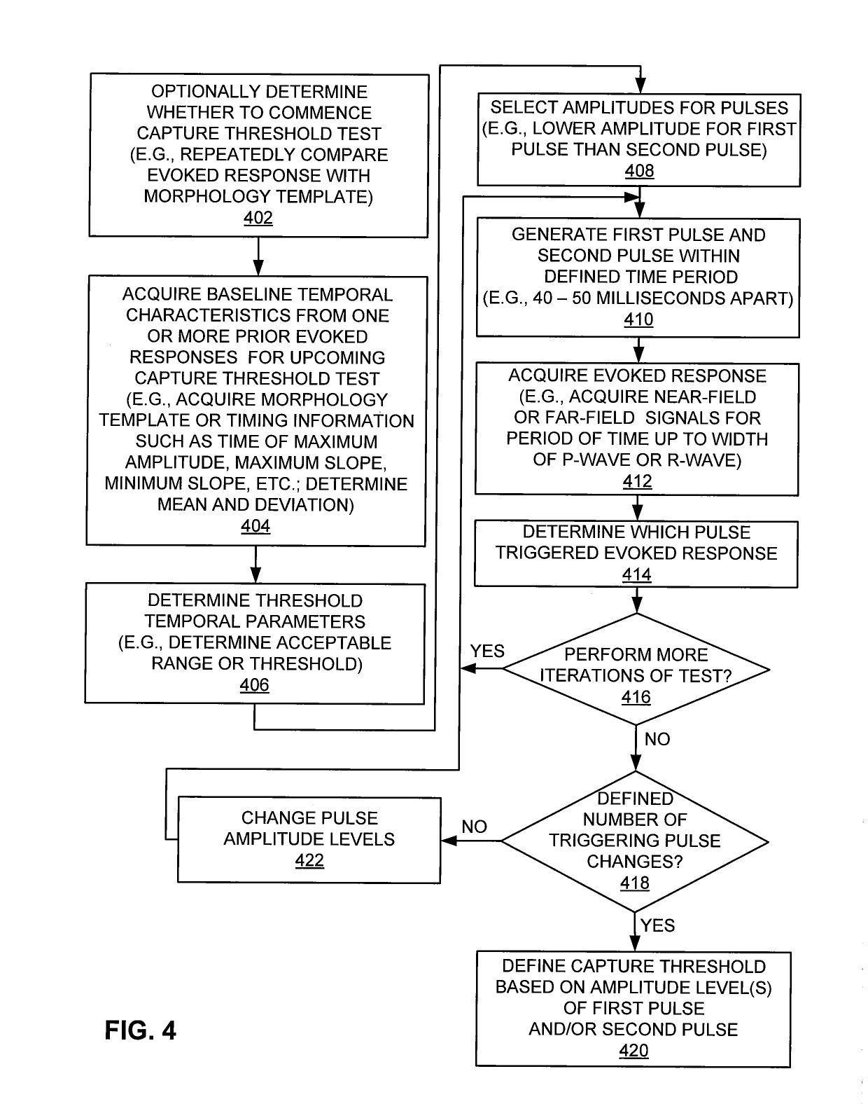

With the above overview in mind, various capture threshold test operations will be discussed in more detail in conjunction with the flowchart of FIG. 4. For convenience, the operations of FIG. 4 (or any other operations discussed or taught herein) may be described as being performed by specific components (e.g., the system 200). It should be appreciated, however, that these operations may be performed in conjunction with and/or by other components and, in some cases, using a different number of components. It also should be appreciated that one or more of these operations may not be employed in a given implementation.

In a typical implementation an implantable cardiac device will invoke a capture threshold test on a regular basis. For example, a capture threshold test may be invoked several times a day at designated times and/or under designated conditions.

As represented by block 402 in FIG. 4, in some embodiments a capture threshold test may be invoked based on certain conditions associated with ongoing pacing operations. For example, the implantable cardiac device may analyze the evoked response (e.g., on a repetitive basis) to determine whether one or more circumstances relating to the evoked response (e.g., lack of consistent capture) indicate that the capture threshold may need to be reset. In some embodiments a morphology template may be used for this purpose. For example, a morphology of a current evoked response may be compared with a morphology template of a typical evoked response to determine whether the current evoked response still correlates well with a typical evoked response.

The implantable cardiac device may generate or otherwise obtain a morphology template using any suitable method such as those that are known in the art. For example, in the system 200 of FIG. 2 evoked response signals 210 acquired by the signal acquisition circuit 206 may be provided to a temporal characteristic acquisition component 212 that comprises a morphology template generator 214. Here, the morphology template generator 214 may collect evoked response information over time (e.g., over several beats) to generate one or more morphology templates. In a typical implementation, a morphology template generator 214 generates an ensemble average of the evoked response signals. In addition, the morphology template generator 214 may perform a cross-correlation over the collected evoked response information to determine the variability of the information (e.g., standard deviation). Resulting morphology template information 220 may then be stored in a data memory 216.

As represented by block 404 in FIG. 4, in some embodiments baseline temporal characteristics of prior evoked responses may be acquired during a period of time that substantially precedes (e.g., immediately precedes) commencement of a capture threshold test. For example, this information may be derived from the evoked responses that occurred during the 20 to 30 beats that immediately precede the capture threshold test. In this way, the characteristics (e.g., morphology and timing) of the baseline information may more closely match the evoked response characteristics that will be acquired during the capture threshold test. In some implementations the recently collected information may be combined with previously collected information to provide the baseline information.

In the example of FIG. 2, the signal acquisition circuit 206 may acquire these evoked response signals at times designated by a timer component 244 or by some other suitable component. For example, the timer component 244 may designate regular intervals at which evoked response signals are acquired, or may trigger evoked response signal acquisition an appropriate period of time (e.g., one minute) before the start of a capture threshold test.

The signal acquisition circuit 206 provides the evoked response information 210 (e.g., an IEGM) to the acquisition component 212 which then processes the information to generate the morphology template and/or other baseline information. As discussed above, the morphology template generator 214 may generate one or more morphology templates to be used in the capture threshold test. In conjunction with this template information, the morphology template generator 214 may determine an acceptable or typical range of tolerance (e.g., standard deviation) associated with the various evoked responses that were processed to obtain the template(s).

The acquisition component 212 also may comprise a timing derivation component 218 that processes the evoked response information 210 (e.g., IEGM) to provide baseline timing information. For example, the timing derivation component 218 may determine the times (e.g., average times) of various features of the evoked response with respect to the time of the corresponding pacing pulse. These time values may include, for example, the time of maximum amplitude, the time of maximum slope, and the time of minimum slope. The timing derivation component 218 also may determine an acceptable or typical range of tolerance (e.g., standard deviation) for these time values based on deviations of the corresponding time values of the various evoked responses that were processed to obtain the baseline time values.

As discussed above, the timing of the current evoked response depends on which pulse captured. Accordingly, the components 214 and 218 may generate different sets of information representative of different evoked responses that are triggered by a first pacing pulse (e.g., test pulse), a second pacing pulse (e.g., backup pulse), etc. For example, in some embodiments the template generator 214 generates a single morphology template wherein different portions (e.g., time periods) within the template are representative of the morphologies that would be generated as a result of capture by the different pulses. Alternatively, in some embodiments the template generator 214 generates a separate template corresponding to each type of pulse. In this case, each template includes information relating to the morphology that may be generated upon capture by the corresponding pulse, taking into account any blanking period (e.g., as shown in FIG. 3B). Similarly, for the baseline time values, the timing derivation component 218 may generate a first set of time values for the first pacing pulse and a second set of time values for the second pacing pulse.

The acquisition component 212 may then store the resulting baseline temporal characteristics in the data memory 216. For example, the morphology templates 220 may include a template 222 for a first pulse and a template 224 for a second pulse. Similarly, the baseline time values 226 may include time values 228 for a first pulse and time values 230 for a second pulse.

As represented by block 406 in FIG. 4, in some embodiments provisions may be made to ensure that the evoked response information acquired during the capture threshold test is sufficiently similar to the template and other baseline information. For example, threshold temporal parameters may be defined such that if the difference between the current evoked response information and typical evoked response information falls outside of an acceptable range (e.g. below a threshold value), the current evoked response information will be ignored for purposes of determining a capture threshold. Such a procedure may be used, for example, to distinguish a fusion beat from a true evoked response.

In the example of FIG. 2 these operations may be performed by the acquisition component 212. The acquisition component 212 may store resulting threshold parameters 232 in the data memory 216. For example, the threshold parameters 232 may include one or more thresholds 234 for a morphology-based test and one or more thresholds 236 for a time value-based test.

For a morphology-based test the acquisition component 212 may generate a threshold parameter such as a minimum correlation score or some other suitable parameter. A minimum correlation score may be based on, for example, an average correlation value derived from a set of evoked responses and an average deviation of those correlation values.

For a time value-based test, the acquisition component 212 may generate a threshold parameter defining a range of acceptable time values (e.g., defined by minimum and maximum time value thresholds) or some other suitable parameter. A range of time values may be based on, for example, an average time value derived from a set of evoked responses and an average deviation of those time values.

In either case, the threshold temporal parameters may be defined to take into account variations of the evoked responses to be tested. For example, a threshold temporal parameter may have a sufficient level of tolerance to account for evoked responses that are triggered by either a test pulse or a backup pulse.

As represented by block 408 in FIG. 4, when the capture threshold test commences the implantable cardiac device may select the initial amplitude levels to be used for each pacing pulse. These levels may be defined, for example, in the manner discussed above in conjunction with block 102. Thus, the amplitude of a backup pulse may be set to a level that is expected to capture, while the test pulse is set to a lower amplitude level.

As represented by block 410, the pulse generator 202 generates the pacing pulses separated in time by a defined time period. For example, as discussed above the pulses may be separated in time by no more than an A-V delay (e.g., less than or equal to 120 milliseconds). Moreover, in a typical implementation the pulses may be separated by a time period on the order of 40-50 milliseconds.

The teaching herein may be employed in conjunction with a capture threshold test for pacing any area of a patient's heart. Consequently, the pacing pulses may be applied, via one or more implanted cardiac leads, to myocardial tissue of the right atrium, the left atrium, the right ventricle, the left ventricle, the pericardium or any other suitable area.

As represented by block 412, the signal acquisition circuit 206 acquires any evoked response that was triggered by the pacing pulses. In a typical implementation, the evoked response may be acquired by sensing from the same implantable lead, and optionally using the same electrodes, that was used to generate the pacing pulses. It should be appreciated, however, that various sensing techniques including detection of near-field signals and/or far-field signals may be employed. As an example of the later scenario, an R-wave may be sensed in an atrial chamber to identify a ventricular pacing pulse that achieved capture. In another example, an indirect evoked response in the form of an R-wave may be sensed in an atrial chamber to determine that the atrial pacing pulse that caused the R-wave achieved capture.

As discussed above in conjunction with FIG. 3, the signal acquisition circuit 206 may acquire a portion or all of an evoked response signal. For example, the signal acquisition circuit 206 may acquire a leading edge, a selected segment, selected segments, or substantially all of a P-wave or an R-wave.

As represented by block 414, the implantable cardiac device processes the acquired evoked response information (e.g., IEGM) to determine which pulse triggered the current evoked response. In conjunction with this operation, a component 238 of the system 200 of FIG. 2 may process the evoked response information to provide one or more temporal characteristics of the evoked response that will be compared with the baseline temporal information. For example, the component 238 may comprise a morphology-based component 240 that derives morphology information from the current evoked response. Similarly, the component 238 may comprise a timing-based component 242 that derives time value information (e.g., a time of maximum amplitude, etc.) from the current evoked response.

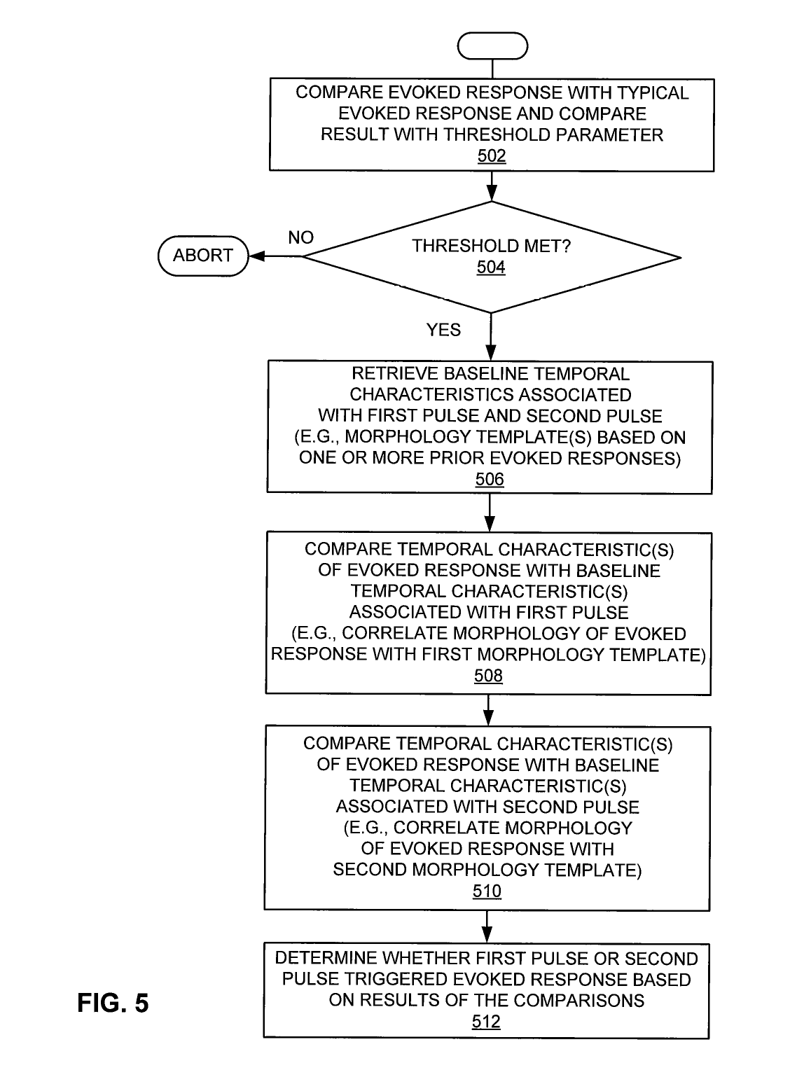

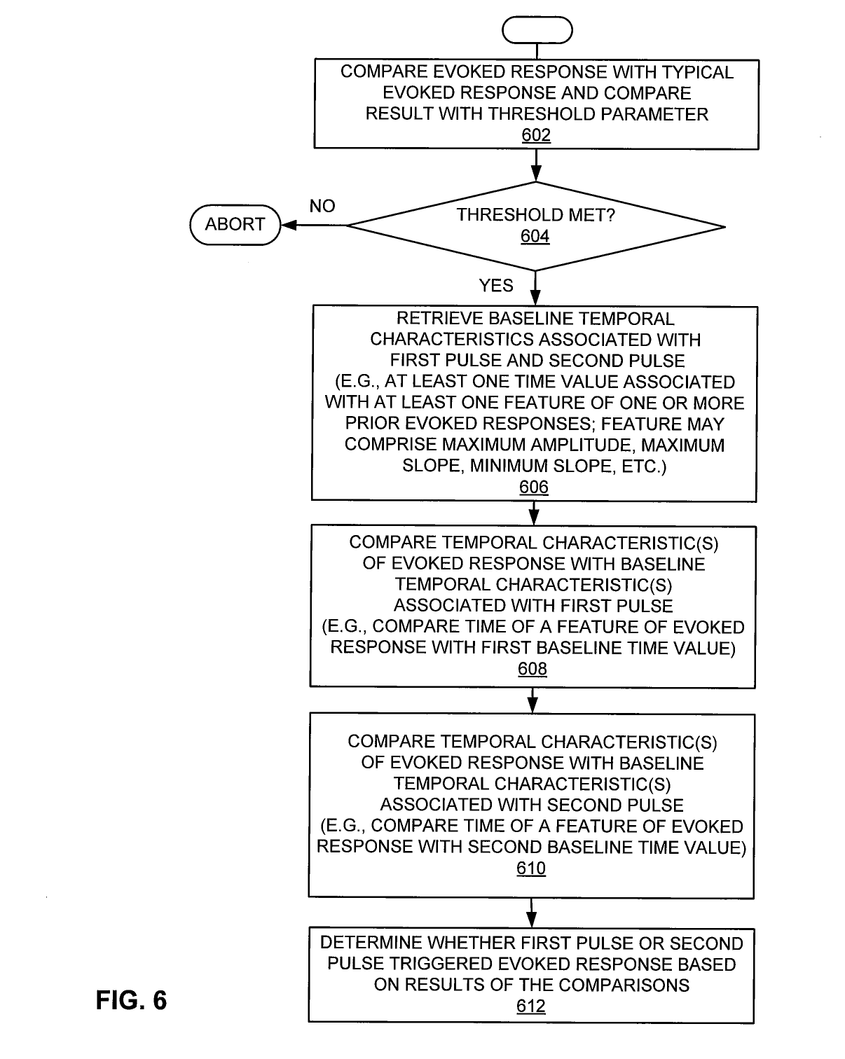

The operations of blocks 414 will be discussed in more detail in conjunction with the flowcharts of FIGS. 5 and 6. FIG. 5 relates to operations that may be performed in embodiments that utilize morphology discrimination to determine whether a first pulse (e.g., a test pulse) or a second pulse (e.g., a backup pulse) triggered the evoked response. FIG. 6 relates to operations that may be performed in embodiments that utilize other baseline temporal information to determine whether the first pulse or the second pulse triggered the evoked response. It should be appreciated that the operations of FIGS. 5 and 6 are but a few examples of techniques that may be employed to determine which pulse triggered an evoked response, and that the teachings herein may be used in conjunction with other suitable techniques.

As represented by block 502 in FIG. 5, initially the current evoked response may be compared with a typical evoked response to determine whether an adequate evoked response signal has been acquired. For example, morphology information representative of the current evoked response (e.g., as provided by component 238) may be compared with morphology information representative of a typical (e.g., average) evoked response. When the comparison involves correlation, a resulting correlation score may be compared with a threshold parameter 234 in the form of a template correlation score (e.g., an average correlation score adjusted according to a desired tolerance). As represented by block 504, in the event the current evoked response is not sufficiently similar to the template, the current evoked response may not be utilized to determine the capture threshold. In this case the processing of the current evoked response may be aborted and another iteration of the capture threshold test may be initiated to acquire and process a different evoked response.

In alternative embodiments, results of each comparison of morphology information for the current evoked response with morphology information for typical evoked responses may, in turn, be compared with a threshold parameter 234 to determine whether the current evoked response is to be used to determine the capture threshold. For example, each comparison result from block 508 and block 510 discussed below may be compared with the threshold parameter 234. Again, the threshold parameter 234 may define an acceptable (e.g., minimum) morphology score.

As represented by block 506, in the event the threshold parameter test of blocks 502 and 504 is met the implantable cardiac device retrieves baseline temporal characteristics associated with the first pulse and the second pulse. As discussed above, this information may take the form of one or more morphology templates 220 that are based on one or more prior evoked responses.

As represented by block 508, the implantable cardiac device then compares one or more temporal characteristics associated with the current evoked response with the baseline temporal characteristics associated with the first pulse. In the example of FIG. 2, a comparator 246 compares the morphology information representative of the current evoked response as provided by the component 238 with morphology information from one of the morphology templates 220. As discussed above, the morphology information associated with the first pulse may comprise a portion of an overall morphology template or may comprise a dedicated morphology template 222. In the former case, a window defined within the overall template may be used to obtain the morphology information associated with the first pulse from the overall template.

In some embodiments the comparison operation comprises a correlation-related operation (e.g., a cross-correlation). To this end, the comparator 246 may comprise a correlator (e.g., a cross-correlator) 248.

As represented by block 510, the implantable cardiac device also compares the temporal characteristic(s) associated with the current evoked response with the baseline temporal characteristics associated with the second pulse. Thus, the comparator 246 may again compare (e.g., correlate) the morphology information representative of the current evoked response as provided by the component 238 with morphology information from one of the morphology templates 220. As discussed above, the morphology information associated with the second pulse may comprise a portion of an overall morphology template or may comprise a dedicated morphology template 224. In the former case, another window defined within the overall template may be used to obtain the morphology information associated with the second pulse.

As represented by block 512, a component 250 may determine whether the first pulse or the second pulse triggered the evoked response. For example, if the comparison result of block 508 indicates a better match (e.g., a higher correlation score) than the comparison result of block 510, the component 250 may determine that the first pulse triggered the evoked response. Referring to the example of FIG. 3, this operation may thus determine that the evoked response more closely correlates with the waveform 306B within the detection window 308B than it correlates with the waveform 306A within detection window 308A.

Referring now to the operations of FIG. 6, as represented by block 602, initially temporal characteristics derived from the current evoked response (e.g., as provided by component 238) may be compared with typical temporal characteristics derived from a typical evoked response to determine whether an adequate evoked response signal has been acquired. Here, the typical temporal characteristics may be derived from an average of such characteristics over several evoked responses, adjusted according to a standard deviation of the characteristics. The result of the comparison may then be compared with a corresponding threshold parameter 236. Here, the threshold parameter 236 may define an acceptable difference between a current time value and a baseline time value, where the baseline time value is based on a previously determined average difference of these values and a desired tolerance. As represented by block 604, in the event the current evoked response information is not sufficiently similar to the baseline information, the current evoked response may not be utilized to determine the capture threshold. Accordingly, the processing of the current evoked response may be aborted and another iteration of the capture threshold test invoked.

In alternative embodiments, results of each comparison of time values derived from the current evoked response with time values derived from the typical evoked responses may, in turn, be compared with a threshold parameter 236 to determine whether the current evoked response is to be used to determine the capture threshold. For example, each comparison result from block 608 and from block 610 discussed below may be compared with the threshold parameter 236.

As represented by block 606, in the event the threshold parameter test of blocks 602 and 604 is met, the implantable cardiac device retrieves baseline temporal characteristics associated with the first pulse and the second pulse. In this case, this information may take the form of one or more baseline time values 226 that are derived from one or more prior evoked responses.

As represented by block 608, the implantable cardiac device then compares one or more temporal characteristics associated with the current evoked response with the baseline temporal characteristics associated with the first pulse. In FIG. 2, the comparator 246 compares the time values derived from the current evoked response as provided by the component 238 with the baseline time values 228 for the first pulse.

As represented by block 610, the implantable cardiac device also compares the temporal characteristic(s) associated with the current evoked response with the baseline temporal characteristics associated with the second pulse. Here, the comparator 246 may compare the time values derived from the current evoked response as provided by the component 238 with the baseline time values 230 for the second pulse.

As represented by block 612, the component 250 may determine whether the first pulse or the second pulse triggered the evoked response. For example, if one or more comparison results of block 604 indicate a better match than the corresponding comparison result(s) of block 606, the component 250 may determine that the first pulse triggered the evoked response. Referring to the example of FIG. 3, this operation may thus determine that the time of the maximum amplitude (and/or some other feature) of the evoked response more closely matches the time of the maximum amplitude of the waveform 306B within the detection window 308B than it matches the time of the maximum amplitude of the waveform 306A within the detection window 308A. In practice, the determination of block 612 may be based on the timing information associated with one of the features of the evoked response or several of these features (e.g., maximum amplitude, maximum slope, minimum slope, some other feature, or some combination of these features).

Based on the results of the determination made at either block 512 or block 612, a component 252 may determine the capture threshold or may modify the current values of the pulse amplitudes 254 to conduct one or more additional iterations of the capture threshold test. Referring now to blocks 416 and 418 in FIG. 4, a determination as to the capture threshold may be made based on whether, in one or more current iterations of the capture threshold test, a different pulse has triggered the evoked response than in the previous iteration of the capture threshold test. As represented by block 416, in some implementations this determination may be made based on the result of multiple iterations of the test. In this way, any adverse effect of noise or some other factor on a given iteration of the test may be mitigated. For example, the test at block 418 may involve determining whether a different pulse has triggered the evoked response a defined number of times (e.g., two out of three times, two times in a row, or some other percentage or number of consecutive beats). Thus, as represented by block 416, in some cases a defined number of iterations of the test may be performed. In some aspects, the operation of block 418 may involve determining whether the pulse that triggered the evoked response has just switched from the first pulse to the second pulse, or vice versa. For example, if the amplitude of the test pulse was being decreased and in the prior iterations of the capture threshold test the test pulse triggered the evoked response, a failure of the test pulse to capture in the current iteration of the test indicates that the amplitude of the test pulse in the previous iteration of the test may provide at least a preliminary value for defining the capture threshold. Conversely, if the amplitude of the test pulse was being increased and in the prior iterations of the capture threshold test the test pulse did not trigger the evoked response, capture by the test pulse in the current iteration of the test indicates that the current amplitude of the test pulse may provide a least a preliminary value for defining the capture threshold. Accordingly, at block 420 the capture threshold may be defined based on a current or prior amplitude level of the first pulse, and in some situations based on the amplitude of the second pulse.

In contrast, in the event there was no change in the triggering pulse at block 418 (or no changes associated with a sufficient number of the pulses), the amplitude level of the first pulse, and optionally the second pulse, may be changed for the next iteration of the capture threshold test (block 422). The operations of blocks 410 to 422 may then be repeated as necessary until a capture threshold is determined.

In view of the above it should be appreciated that one or more advantages may be achieved through the use of the teachings herein. For example, backup pacing may always be provided with minimal disruption of the normal cardiac rhythm. A relatively large detection window may be used for capture detection, thereby enabling more robust evoked response analysis. For ventricle pacing, the paced QRST complex may be used rather than just the leading edge of the paste evoked response (e.g., as may be used for conventional PDI and DMAX operations) thereby providing more tolerance for small or slow evoked response signals and enabling the system to be more tolerant of polarization effects.

### Exemplary Cardiac Device

As mentioned above, the teachings herein may be implemented in an implantable cardiac device. The following describes an example of an implantable cardiac device (e.g., a stimulation device such as an implantable cardioverter defibrillator, a pacemaker, etc.) that is capable of being used in connection with the various embodiments that are described herein. It is to be appreciated and understood that other cardiac devices, including those that are not necessarily implantable, can be used and that the description below is given, in its specific context, to assist the reader in understanding, with more clarity, the embodiments described herein.

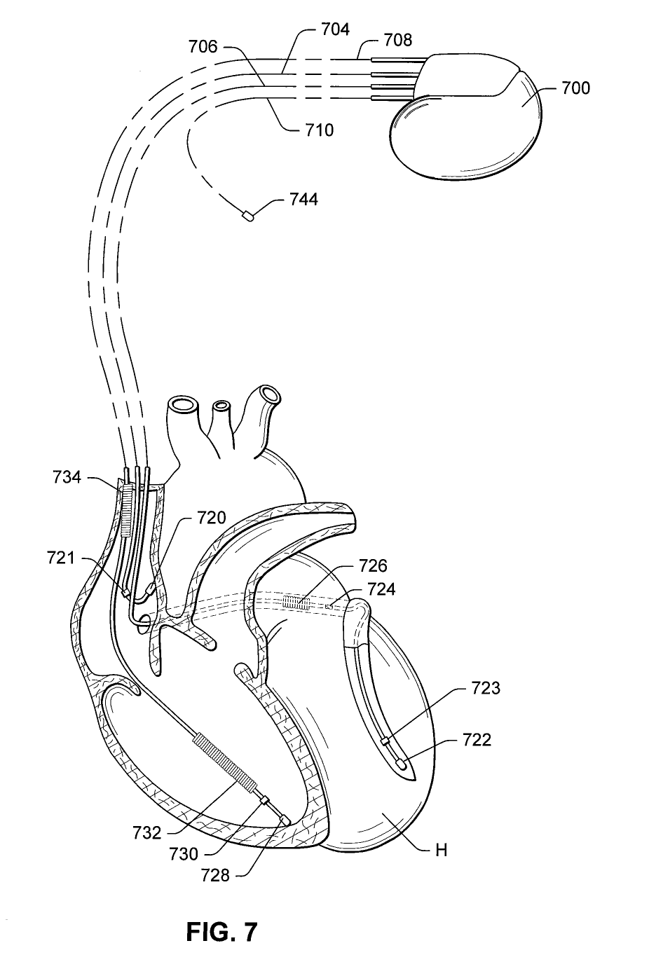

FIG. 7 depicts an embodiment of an implantable cardiac device 700 in electrical communication with a patient's heart H by way of three leads 704, 706, and 708, suitable for delivering multi-chamber stimulation and shock therapy. To sense atrial cardiac signals and to provide right atrial chamber stimulation therapy, the device 700 is coupled to an implantable right atrial lead 704 having, for example, an atrial tip electrode 720, which typically is implanted in the patient's right atrial appendage or septum. FIG. 7 also shows the right atrial lead 704 as having an optional atrial ring electrode 721.

To sense left atrial and ventricular cardiac signals and to provide left chamber pacing therapy, the device 700 is coupled to a coronary sinus lead 706 designed for placement in the coronary sinus region via the coronary sinus for positioning one or more electrodes adjacent to the left ventricle, one or more electrodes adjacent to the left atrium, or both. As used herein, the phrase “coronary sinus region” refers to the vasculature of the left ventricle, including any portion of the coronary sinus, the great cardiac vein, the left marginal vein, the left posterior ventricular vein, the middle cardiac vein, the small cardiac vein or any other cardiac vein accessible by the coronary sinus.

Accordingly, an exemplary coronary sinus lead 706 is designed to receive atrial and ventricular cardiac signals and to deliver left ventricular pacing therapy using, for example, a left ventricular tip electrode 722 and, optionally, a left ventricular ring electrode 723; provide left atrial pacing therapy using, for example, a left atrial ring electrode 724; and provide shocking therapy using, for example, a left atrial coil electrode 726 (or other electrode capable of delivering a shock). For a more detailed description of a coronary sinus lead, the reader is directed to U.S. Pat. No. 5,466,254, “Coronary Sinus Lead with Atrial Sensing Capability” (Helland), which is incorporated herein by reference.

The device 700 is also shown in electrical communication with the patient's heart H by way of an implantable right ventricular lead 708 having, in this implementation, a right ventricular tip electrode 728, a right ventricular ring electrode 730, a right ventricular (RV) coil electrode 732 (or other electrode capable of delivering a shock), and a superior vena cava (SVC) coil electrode 734 (or other electrode capable of delivering a shock). Typically, the right ventricular lead 708 is transvenously inserted into the heart H to place the right ventricular tip electrode 728 in the right ventricular apex so that the RV coil electrode 732 will be positioned in the right ventricle and the SVC coil electrode 734 will be positioned in the superior vena cava. Accordingly, the right ventricular lead 708 is capable of sensing or receiving cardiac signals, and delivering stimulation in the form of pacing and shock therapy to the right ventricle.

The device 700 is also shown in electrical communication with a lead 710 including one or more components 744 such as a physiologic sensor. The component 744 may be positioned in, near or remote from the heart.

It should be appreciated that the device 700 may connect to leads other than those specifically shown. In addition, the leads connected to the device 700 may include components other than those specifically shown. For example, a lead may include other types of electrodes, sensors or devices that serve to otherwise interact with a patient or the surroundings.

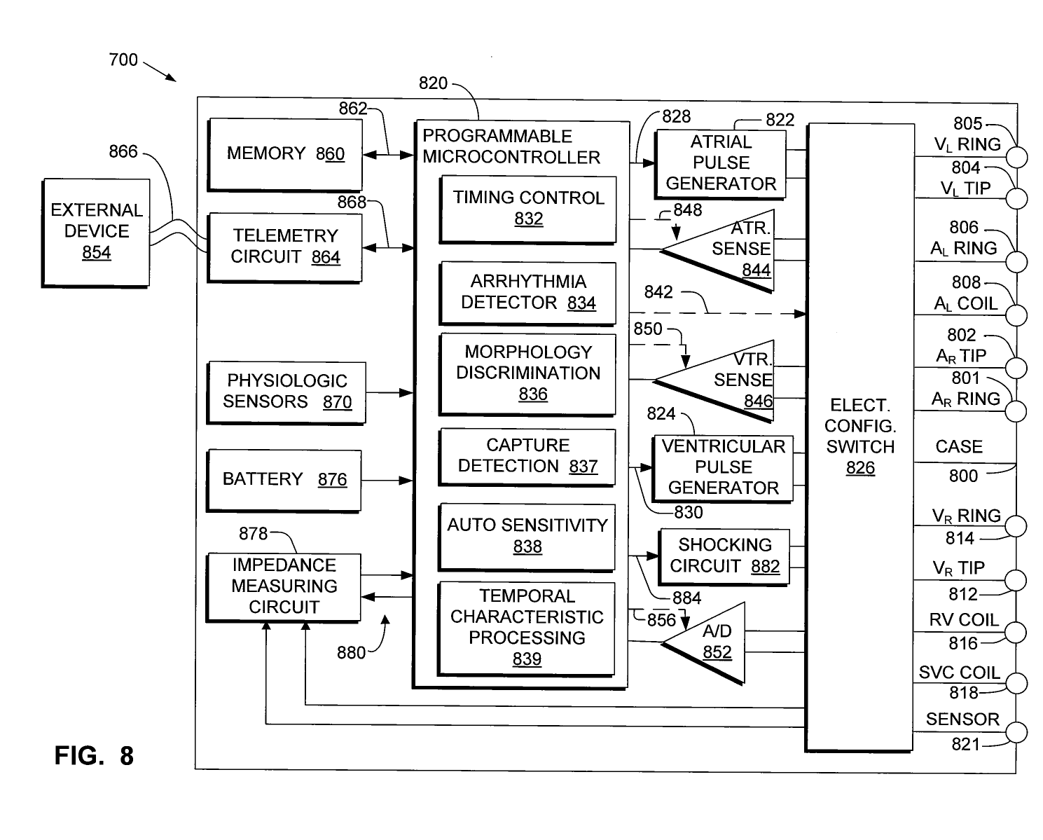

FIG. 8 depicts an exemplary, simplified block diagram illustrating sample components of the device 700. The device 700 may be adapted to treat both fast and slow arrhythmias with stimulation therapy, including cardioversion, defibrillation, and pacing stimulation. While a particular multi-chamber device is shown, it is to be appreciated and understood that this is done for illustration purposes. Thus, the techniques and methods described below can be implemented in connection with any suitably configured or configurable device. Accordingly, one of skill in the art could readily duplicate, eliminate, or disable the appropriate circuitry in any desired combination to provide a device capable of treating the appropriate chamber(s) with, for example, cardioversion, defibrillation, and pacing stimulation.

The housing 800 for the device 700 is often referred to as the “can”, “case” or “case electrode”, and may be programmably selected to act as the return electrode for all “unipolar” modes. The housing 800 may further be used as a return electrode alone or in combination with one or more of the coil electrodes 726, 732 and 734 for shocking purposes. The housing 800 further includes a connector (not shown) having a plurality of terminals 801, 802, 804, 805, 806, 808, 812, 814, 816 and 818 (shown schematically and, for convenience, the names of the electrodes to which they are connected are shown next to the terminals). The connector may be configured to include various other terminals depending on the requirements of a given application.

To achieve right atrial sensing and pacing, the connector includes, for example, a right atrial tip terminal (AR TIP) 802 adapted for connection to the right atrial tip electrode 720. A right atrial ring terminal (AR RING) 801 may also be included and adapted for connection to the right atrial ring electrode 721. To achieve left chamber sensing, pacing, and shocking, the connector includes, for example, a left ventricular tip terminal (VL TIP) 804, a left ventricular ring terminal (VL RING) 805, a left atrial ring terminal (AL RING) 806, and a left atrial shocking terminal (AL COIL) 808, which are adapted for connection to the left ventricular tip electrode 722, the left ventricular ring electrode 723, the left atrial ring electrode 724, and the left atrial coil electrode 726, respectively.

To support right chamber sensing, pacing, and shocking, the connector further includes a right ventricular tip terminal (VR TIP) 812, a right ventricular ring terminal (VR RING) 814, a right ventricular shocking terminal (RV COIL) 816, and a superior vena cava shocking terminal (SVC COIL) 818, which are adapted for connection to the right ventricular tip electrode 728, the right ventricular ring electrode 730, the RV coil electrode 732, and the SVC coil electrode 734, respectively.

At the core of the device 700 is a programmable microcontroller 820 that controls the various modes of stimulation therapy. As is well known in the art, microcontroller 820 typically comprises a microprocessor, or equivalent control circuitry, designed specifically for controlling the delivery of stimulation therapy, and may further include memory such as RAM, ROM and flash memory, logic and timing circuitry, state machine circuitry, and I/O circuitry. Typically, microcontroller 820 includes the ability to process or monitor input signals (data or information) as controlled by a program code stored in a designated block of memory. The type of microcontroller is not critical to the described implementations. Rather, any suitable microcontroller 820 may be used that carries out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052 (Mann et al.), the state-machine of U.S. Pat. No. 4,712,555 (Thornander et al.) and U.S. Pat. No. 4,944,298 (Sholder), all of which are incorporated by reference herein. For a more detailed description of the various timing intervals that may be used within the device and their inter-relationship, see U.S. Pat. No. 4,788,980 (Mann et al.), also incorporated herein by reference.

FIG. 8 also shows an atrial pulse generator 822 and a ventricular pulse generator 824 that generate pacing stimulation pulses for delivery by the right atrial lead 704, the coronary sinus lead 706, the right ventricular lead 708, or some combination of these leads via an electrode configuration switch 826. It is understood that in order to provide stimulation therapy in each of the four chambers of the heart, the atrial and ventricular pulse generators 822 and 824 may include dedicated, independent pulse generators, multiplexed pulse generators, or shared pulse generators. The pulse generators 822 and 824 are controlled by the microcontroller 820 via appropriate control signals 828 and 830, respectively, to trigger or inhibit the stimulation pulses.

Microcontroller 820 further includes timing control circuitry 832 to control the timing of the stimulation pulses (e.g., pacing rate, atrio-ventricular (A-V) delay, atrial interconduction (A-A) delay, or ventricular interconduction (V-V) delay, etc.) or other operations, as well as to keep track of the timing of refractory periods, blanking intervals, noise detection windows, evoked response windows, alert intervals, marker channel timing, etc., as known in the art.

Microcontroller 820 further includes an arrhythmia detector 834. The arrhythmia detector 834 may be utilized by the device 700 for determining desirable times to administer various therapies. The arrhythmia detector 834 may be implemented, for example, in hardware as part of the microcontroller 820, or as software/firmware instructions programmed into the device 700 and executed on the microcontroller 820 during certain modes of operation.

Microcontroller 820 may include a morphology discrimination module 836, a capture detection module 837 and an auto sensing module 838. These modules are optionally used to implement various exemplary recognition algorithms or methods. The aforementioned components may be implemented, for example, in hardware as part of the microcontroller 820, or as software/firmware instructions programmed into the device 700 and executed on the microcontroller 820 during certain modes of operation.

The electrode configuration switch 826 includes a plurality of switches for connecting the desired terminals (e.g., that are connected to electrodes, coils, sensors, etc.) to the appropriate I/O circuits, thereby providing complete terminal and, hence, electrode programmability. Accordingly, switch 826, in response to a control signal 842 from the microcontroller 820, may be used to determine the polarity of the stimulation pulses (e.g., unipolar, bipolar, combipolar, etc.) by selectively closing the appropriate combination of switches (not shown) as is known in the art.

Atrial sensing circuits (ATR. SENSE) 844 and ventricular sensing circuits (VTR. SENSE) 846 may also be selectively coupled to the right atrial lead 704, coronary sinus lead 706, and the right ventricular lead 708, through the switch 826 for detecting the presence of cardiac activity in each of the four chambers of the heart. Accordingly, the atrial and ventricular sensing circuits 844 and 846 may include dedicated sense amplifiers, multiplexed amplifiers, or shared amplifiers. Switch 826 determines the “sensing polarity” of the cardiac signal by selectively closing the appropriate switches, as is also known in the art. In this way, the clinician may program the sensing polarity independent of the stimulation polarity. The sensing circuits (e.g., circuits 844 and 846) are optionally capable of obtaining information indicative of tissue capture.

Each sensing circuit 844 and 846 preferably employs one or more low power, precision amplifiers with programmable gain, automatic gain control, bandpass filtering, a threshold detection circuit, or some combination of these components, to selectively sense the cardiac signal of interest. The automatic gain control enables the device 700 to deal effectively with the difficult problem of sensing the low amplitude signal characteristics of atrial or ventricular fibrillation.

The outputs of the atrial and ventricular sensing circuits 844 and 846 are connected to the microcontroller 820, which, in turn, is able to trigger or inhibit the atrial and ventricular pulse generators 822 and 824, respectively, in a demand fashion in response to the absence or presence of cardiac activity in the appropriate chambers of the heart. Furthermore, as described herein, the microcontroller 820 is also capable of analyzing information output from the sensing circuits 844 and 846, a data acquisition system 852, or both. This information may be used to determine or detect whether and to what degree tissue capture has occurred and to program a pulse, or pulses, in response to such determinations. The sensing circuits 844 and 846, in turn, receive control signals over signal lines 848 and 850, respectively, from the microcontroller 820 for purposes of controlling the gain, threshold, polarization charge removal circuitry (not shown), and the timing of any blocking circuitry (not shown) coupled to the inputs of the sensing circuits 844 and 846 as is known in the art.

For arrhythmia detection, the device 700 utilizes the atrial and ventricular sensing circuits 844 and 846 to sense cardiac signals to determine whether a rhythm is physiologic or pathologic. It should be appreciated that other components may be used to detect arrhythmia depending on the system objectives. In reference to arrhythmias, as used herein, “sensing” is reserved for the noting of an electrical signal or obtaining data (information), and “detection” is the processing (analysis) of these sensed signals and noting the presence of an arrhythmia.

Timing intervals between sensed events (e.g., P-waves, R-waves, and depolarization signals associated with fibrillation) may be classified by the arrhythmia detector 834 of the microcontroller 820 by comparing them to a predefined rate zone limit (e.g., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”). Similar rules may be applied to the atrial channel to determine if there is an atrial tachyarrhythmia or atrial fibrillation with appropriate classification and intervention.

Cardiac signals or other signals may be applied to inputs of an analog-to-digital (A/D) data acquisition system 852. The data acquisition system 852 is configured (e.g., via signal line 856) to acquire intracardiac electrogram (“IEGM”) signals or other signals, convert the raw analog data into a digital signal, and store the digital signals for later processing, for telemetric transmission to an external device 854, or both. For example, the data acquisition system 852 may be coupled to the right atrial lead 704, the coronary sinus lead 706, the right ventricular lead 708 and other leads through the switch 826 to sample cardiac signals across any pair of desired electrodes.

The data acquisition system 852 also may be coupled to receive signals from other input devices. For example, the data acquisition system 852 may sample signals from a physiologic sensor 870 or other components shown in FIG. 8 (connections not shown).

The microcontroller 820 is further coupled to a memory 860 by a suitable data/address bus 862, wherein the programmable operating parameters used by the microcontroller 820 are stored and modified, as required, in order to customize the operation of the device 700 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart H within each respective tier of therapy. One feature of the described embodiments is the ability to sense and store a relatively large amount of data (e.g., from the data acquisition system 852), which data may then be used for subsequent analysis to guide the programming of the device 700.

Advantageously, the operating parameters of the implantable device 700 may be non-invasively programmed into the memory 860 through a telemetry circuit 864 in telemetric communication via communication link 866 with the external device 854, such as a programmer, transtelephonic transceiver, a diagnostic system analyzer or some other device. The microcontroller 820 activates the telemetry circuit 864 with a control signal (e.g., via bus 868). The telemetry circuit 864 advantageously allows intracardiac electrograms and status information relating to the operation of the device 700 (as contained in the microcontroller 820 or memory 860) to be sent to the external device 854 through an established communication link 866.

The device 700 can further include one or more physiologic sensors 870. In some embodiments the device 700 may include a “rate-responsive”sensor that may provide, for example, information to aid in adjustment of pacing stimulation rate according to the exercise state of the patient. One or more physiologic sensors 870 (e.g., a pressure sensor) may further be used to detect changes in cardiac output, changes in the physiological condition of the heart, or diurnal changes in activity (e.g., detecting sleep and wake states). Accordingly, the microcontroller 820 responds by adjusting the various pacing parameters (such as rate, AV Delay, V-V Delay, etc.) at which the atrial and ventricular pulse generators 822 and 824 generate stimulation pulses.

While shown as being included within the device 700, it is to be understood that a physiologic sensor 870 may also be external to the device 700, yet still be implanted within or carried by the patient. Examples of physiologic sensors that may be implemented in conjunction with the device 700 include sensors that sense respiration rate, pH of blood, ventricular gradient, oxygen saturation, blood pressure and so forth. Another sensor that may be used is one that detects activity variance, wherein an activity sensor is monitored diurnally to detect the low variance in the measurement corresponding to the sleep state. For a more detailed description of an activity variance sensor, the reader is directed to U.S. Pat. No. 5,476,483 (Bornzin et al.), issued Dec. 19, 1995, which patent is hereby incorporated by reference.

The one or more physiologic sensors 870 may optionally include one or more of components to help detect movement (via, e.g., a position sensor or an accelerometer) and minute ventilation (via an MV sensor) in the patient. Signals generated by the position sensor and MV sensor may be passed to the microcontroller 820 for analysis in determining whether to adjust the pacing rate, etc. The microcontroller 820 may thus monitor the signals for indications of the patient's position and activity status, such as whether the patient is climbing up stairs or descending down stairs or whether the patient is sitting up after lying down.

The device 700 additionally includes a battery 876 that provides operating power to all of the circuits shown in FIG. 8. For a device 700 which employs shocking therapy, the battery 876 is capable of operating at low current drains (e.g., preferably less than 10 μA) for long periods of time, and is capable of providing high-current pulses (for capacitor charging) when the patient requires a shock pulse (e.g., preferably, in excess of 2 A, at voltages above 200 V, for periods of 10 seconds or more). The battery 876 also desirably has a predictable discharge characteristic so that elective replacement time can be detected. Accordingly, the device 700 preferably employs lithium or other suitable battery technology.

The device 700 can further include magnet detection circuitry (not shown), coupled to the microcontroller 820, to detect when a magnet is placed over the device 700. A magnet may be used by a clinician to perform various test functions of the device 700 and to signal the microcontroller 820 that the external device 854 is in place to receive data from or transmit data to the microcontroller 820 through the telemetry circuit 864.

The device 700 further includes an impedance measuring circuit 878 that is enabled by the microcontroller 820 via a control signal 880. The known uses for an impedance measuring circuit 878 include, but are not limited to, lead impedance surveillance during the acute and chronic phases for proper performance, lead positioning or dislodgement; detecting operable electrodes and automatically switching to an operable pair if dislodgement occurs; measuring respiration or minute ventilation; measuring thoracic impedance for determining shock thresholds; detecting when the device 700 has been implanted; measuring stroke volume; and detecting the opening of heart valves, etc. The impedance measuring circuit 878 is advantageously coupled to the switch 826 so that any desired electrode may be used.

In the case where the device 700 is intended to operate as an implantable cardioverter/defibrillator (ICD) device, it detects the occurrence of an arrhythmia, and automatically applies an appropriate therapy to the heart aimed at terminating the detected arrhythmia. To this end, the microcontroller 820 further controls a shocking circuit 882 by way of a control signal 884. The shocking circuit 882 generates shocking pulses of low (e.g., up to 0.5 J), moderate (e.g., 0.5 J to 10 J), or high energy (e.g., 11 J to 40 J), as controlled by the microcontroller 820. Such shocking pulses are applied to the patient's heart H through, for example, two shocking electrodes and as shown in this embodiment, selected from the left atrial coil electrode 726, the RV coil electrode 732 and the SVC coil electrode 734. As noted above, the housing 800 may act as an active electrode in combination with the RV coil electrode 732, as part of a split electrical vector using the SVC coil electrode 734 or the left atrial coil electrode 726 (i.e., using the RV electrode as a common electrode), or in some other arrangement.

Cardioversion level shocks are generally considered to be of low to moderate energy level (so as to minimize pain felt by the patient), be synchronized with an R-wave, pertain to the treatment of tachycardia, or some combination of the above. Defibrillation shocks are generally of moderate to high energy level (i.e., corresponding to thresholds in the range of 5 J to 40 J), delivered asynchronously (since R-waves may be too disorganized), and pertaining exclusively to the treatment of fibrillation. Accordingly, the microcontroller 820 is capable of controlling the synchronous or asynchronous delivery of the shocking pulses.

The device 700 includes several components that may provide functionality relating to a capture threshold test. For example, the pulse generators 822 and 824 may generate pacing pulses as discussed herein. In addition, one or more of the sense circuits 844 and 846, the data acquisition system 852, and the switch 826 may provide signal acquisition-related functionality as discussed herein.

The microcontroller 820 (e.g., a processor providing signal processing functionality) also may implement threshold test-related functionality as discussed herein. For example, the microcontroller may comprise a temporal characteristic processing module 839 that derives and processes (e.g. compares) temporal-related information from evoked response signals or other signals for the capture threshold test. The capture detection module 837 may provide capture threshold test-related functionality as discussed herein. In addition, the morphology discrimination module 836 may provide morphology-related functionality for the capture threshold test as discussed herein. Also, the timing control circuitry 832 may provide the timer-related functionality for the capture threshold test as discussed herein.

It should be appreciated that various modifications may be incorporated into the disclosed embodiments based on the teachings herein. For example, the structure and functionality taught herein may be incorporated into different types of devices other than those types specifically described. In addition, various algorithms and techniques other than those specifically mentioned herein may be employed to obtain and process information relating to an evoked response signal or other signals and relating to determination of a cardiac capture threshold.

It should thus be appreciated from the above that the various structures and functions described herein may be incorporated into a variety of apparatuses (e.g., a stimulation device, a lead, a monitoring device, etc.) and implemented in a variety of ways. Different embodiments of the implantable cardiac device may include a variety of hardware and software processing components. In some embodiments, hardware components such a processor, a controller, a state machine, logic, or some combination of these components, may be used to implement one or more of the described components or circuits.

In some embodiments, code including instructions (e.g., software, firmware, middleware, etc.) may be executed on one or more processing devices to implement one or more of the described functions or components. The code and associated components (e.g., data structures and other components by the code or to execute the code) may be stored in an appropriate data memory that is readable by a processing device (e.g., commonly referred to as a computer-readable medium).

Moreover, some of the operations described herein may be performed by a device that is located externally with respect to the body of the patient. For example, an implanted device may simply send raw data or processed data to an external device that then performs the necessary processing.

The components and functions described herein may be connected or coupled in many different ways. The manner in which this is done may depend, in part, on whether and how the components are separated from the other components. In some embodiments some of the connections or couplings represented by the lead lines in the drawings may be in an integrated circuit, on a circuit board or implemented as discrete wires or in other ways.

The signals discussed herein may take various forms. For example, in some embodiments a signal may comprise electrical signals transmitted over a wire, light pulses transmitted through an optical medium such as an optical fiber or air, or RF waves transmitted through a medium such as air, etc. In addition, a plurality of signals may be collectively referred to as a signal herein. The signals discussed above also may take the form of data. For example, in some embodiments an application program may send a signal to another application program. Such a signal may be stored in a data memory.

The recited order of the blocks in the processes disclosed herein is simply an example of a suitable approach. Thus, operations associated with such blocks may be rearranged while remaining within the scope of the present disclosure. Similarly, the accompanying method claims present operations in a sample order, and are not necessarily limited to the specific order presented.

While certain exemplary embodiments have been described above in detail and shown in the accompanying drawings, it is to be understood that such embodiments are merely illustrative of and not restrictive of the broad invention. In particular, it should be recognized that the teachings herein apply to a wide variety of apparatuses and methods. It will thus be recognized that various modifications may be made to the illustrated and other embodiments described above, without departing from the broad inventive scope thereof. In view of the above it will be understood that the invention is not limited to the particular embodiments or arrangements disclosed, but is rather intended to cover any changes, adaptations or modifications which are within the scope of the invention as defined by the appended claims.

What is claimed is:

1. A method of determining a cardiac capture threshold for a patient, comprising:
generating a plurality of cardiac pacing pulses within a period of time that is less than an atrioventricular conduction delay of the patient;
acquiring an evoked response triggered by one of the cardiac pacing pulses; and
determining a capture threshold based on at least one temporal characteristic of the evoked response.

2. The method of claim 1, wherein the determining comprises comparing the at least one temporal characteristic of the evoked response with at least one temporal characteristic associated with at least one prior evoked response to determine which one of the cardiac pacing pulses triggered the evoked response.

3. The method of claim 2, wherein:
the at least one temporal characteristic of the evoked response comprises morphology information; and
the at least one temporal characteristic associated with at least one prior evoked response comprises at least one morphology template.

4. The method of claim 3, wherein the at least one morphology template comprises a template including information:
associated with a first time period that corresponds to an expected timing of an evoked response triggered by a first one of the cardiac pacing pulses; and
associated with a second time period that corresponds to an expected timing of an evoked response triggered by a second one of the cardiac pacing pulses.

5. The method of claim 3, wherein the at least one morphology template comprises:
a first template representative of an expected evoked response triggered by a first one of the cardiac pacing pulses; and
a second template representative of an expected evoked response triggered by a second one of the cardiac pacing pulses.

6. The method of claim 2, wherein:
the at least one temporal characteristic of the evoked response comprises at least one time value associated with at least one feature of the evoked response; and
the at least one temporal characteristic associated with at least one prior evoked response comprises at least one baseline time value.

7. The method of claim 6, wherein the at least one feature comprises at least one of the group consisting of: a maximum amplitude, a maximum slope, and a minimum slope.

8. The method of claim 1, wherein the period of time is less than 60 milliseconds.

9. The method of claim 1, wherein acquiring the evoked response comprises detecting a far-field signal.

10. The method of claim 1, wherein the determining comprises defining the capture threshold based on a lowest amplitude associated with one of the cardiac pacing pulses that resulted in capture.

11. An implantable cardiac apparatus for determining a cardiac capture threshold for a patient, comprising:
a pulse generator adapted to generate a plurality of cardiac pacing pulses within a period of time that is less than an atrioventricular conduction delay of the patient;
a signal acquisition circuit adapted to acquire an evoked response triggered by one of the cardiac pacing pulses; and
a processor adapted to determine a capture threshold based on at least one temporal characteristic of the evoked response.

12. The apparatus of claim 11, wherein:
the processor comprises a comparator adapted to compare the at least one temporal characteristic of the evoked response with at least one temporal characteristic associated with at least one prior evoked response; and
the processor is further adapted to determine which one of the cardiac pacing pulses triggered the evoked response.

13. The apparatus of claim 12, wherein:
the at least one temporal characteristic of the evoked response comprises morphology information;
the at least one temporal characteristic associated with at least one prior evoked response comprises at least one morphology template; and
the processor comprises a morphology template generator adapted to generate the at least one morphology template.

14. The apparatus of claim 13, wherein the at least one morphology template comprises an ensemble average derive from a plurality of prior evoked responses.

15. The apparatus of claim 13, wherein the comparator comprises a correlator adapted to correlate the morphology information with the at least one morphology template.

16. The apparatus of claim 13, wherein the at least one morphology template comprises a template including information:
associated with a first time period that corresponds to an expected timing of an evoked response triggered by a first one of the cardiac pacing pulses; and
associated with a second time period that corresponds to an expected timing of an evoked response triggered by a second one of the cardiac pacing pulses.

17. The apparatus of claim 13, wherein the at least one morphology template comprises:
a first template representative of an expected evoked response triggered by a first one of the cardiac pacing pulses; and
a second template representative of an expected evoked response triggered by a second one of the cardiac pacing pulses.

18. The apparatus of claim 13, further comprising a timer adapted to control acquisition of evoked response information during a period of time that substantially precedes commencement of a capture threshold test, wherein the morphology template generator uses the acquired evoked response information to generate the at least one morphology template.

19. The apparatus of claim 12, wherein:
the at least one temporal characteristic of the evoked response comprises at least one time value associated with at least one feature of the evoked response;
the at least one temporal characteristic associated with at least one prior evoked response comprises at least one baseline time value; and
the processor further comprises a timing derivation module adapted to generate the at least one baseline time value.

20. The apparatus of claim 12, wherein:
the at least one temporal characteristic associated with the at least one prior evoked response comprises a plurality of baseline temporal characteristics, each of which is associated with a unique one of the cardiac pacing pulses; and
the comparator is further adapted to compare the at least one temporal characteristic associated with the evoked response with each of the baseline temporal characteristics to generate a corresponding comparison result for each of the cardiac pacing pulses; and
the processor is further adapted to determine, based on the comparison results, which cardiac pacing pulse triggered the evoked response.
